(19) United States
(12) Patent Application Publication
(10) Pub. No.: US 2024/0207441 A1
(43) Pub. Date: Jun. 27, 2024

(54) CAPSID VARIANTS AND USES THEREOF

(71) Applicant: University of Massachusetts, Boston, MA (US)

(72) Inventors: Yukiko Maeda, Worcester, MA (US); Phillip Tai, Worcester, MA (US); Guangping Gao, Worcester, MA (US)

(73) Assignee: University of Massachusetts, Boston, MA (US)

(21) Appl. No.: 18/288,216

(22) PCT Filed: Apr. 26, 2022
(86) PCT No.: PCT/US2022/026325

§ 371 Date: Oct. 25, 2023

Related U.S. Application Data
(60) Provisional application No. 63/180,002, filed on Apr. 26, 2021.

(51) Int. Cl. A61K 48/00; C07K 14/005; C12N 15/86

(52) U.S. Cl. CPC A61K 48/0041; A61K 48/0075; C07K 14/005; C12N 15/86; C12N 2750/14122; C12N 2750/14143; C12N 2750/14145

(57) ABSTRACT

Aspects of the disclosure relate to compositions and methods for delivering a transgene (e.g., an anti-inflammatory transgene encoding one or more gene products) to a joint. The disclosure is based, in part, on adeno-associated virus (AAV) capsid protein variants characterized by tropisms for joint tissues (e.g., cartilage, skin, muscle, and bone cells). Methods of treating arthritis in a joint by administering an rAAV comprising the AAV capsid protein variants are also described by the disclosure.

### RELATED APPLICATIONS

This application is a national stage filing under 35 U.S.C. § 371 of international PCT application PCT/US2022/026325, filed Apr. 26, 2022, which claims priority under 35 U.S.C. § 119(e) to U.S. provisional patent application, U.S. Ser. No. 63/180,002, filed on Apr. 26, 2021, the entire contents of each of which are incorporated by reference herein.

### REFERENCE TO AN ELECTRONIC SEQUENCE LISTING

The contents of the electronic sequence listing (U012070162US01-SEQ-KZM.txt; Size: 10,540,165 bytes; and Date of Creation: Oct. 17, 2023) is herein incorporated by reference in its entirety.

### BACKGROUND

Arthritis is a leading cause of disability among adults in the United States. In the United States, 23% of all adults, or more than 54 million people, have arthritis with annual costs for medical care and lost earnings of $303.5 billion. Due to increased life expectancy and skyrocketing obesity rates in the US, the number of arthritis patients is expected to increase almost 49% to more than 78 million in 2040. Osteoarthritis (OA) is the most common type of arthritis. Risk factors include excess weight, repeated stress on the joint, age, and previous injury. When the cartilage wears away, bone rubs against bone, causing pain, swelling, and stiffness. Over time, joints can lose strength and pain may become chronic. Currently, there is no way to cure OA or stop its progression, and treatments are aimed only at alleviating the symptoms.

### SUMMARY

Aspects of the disclosure relate to compositions and methods for delivering a transgene (e.g., an anti-inflammatory transgene encoding one or more gene products) to arthritic tissues and joints. The disclosure is based, in part, on adeno-associated virus (AAV) capsid protein variants characterized by tropisms for certain cell types present in arthritic tissues and joints (e.g., skin, muscle, bone cells, articular cartilage, meniscus, synovium, and/or ligament tissues of a joint). In some embodiments, recombinant AAVs (rAAVs) comprising the capsid protein variants are more efficiently packaged than rAAVs having certain wild-type AAV capsid proteins. Methods of delivering an rAAV comprising the AAV capsid protein variants and methods of treating arthritis (e.g., osteoarthritis) are also described by the disclosure.

Some aspects of the disclosure provides a method for delivering a transgene to a joint in a subject. In some embodiments, a method for delivering a transgene to a joint in a subject comprises administering to the subject a recombinant adeno-associated virus (rAAV) comprising: (i) an isolated nucleic acid comprising a transgene encoding one or more gene products of interest; and (ii) an adeno-associated acid (AAV) capsid protein having the sequence set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84.

In some embodiments, the administration comprises intra-articular injection. In some embodiments, the joint is a knee joint, shoulder joint, elbow joint, hip joint, ankle joint, spinal joint, finger joint, or toe joint. In some embodiments, the joint comprises skin, muscle, bone cells, articular cartilage, meniscus, synovium, and/or ligament tissues.

In some embodiments, the subject is a mammal, optionally wherein the mammal is a human. In some embodiments, the subject is characterized by production of anti-AAV2 antibodies (e.g., neutralizing antibodies (NAbs) against AAV2) prior to the administration. In some embodiments, after administration of the rAAV, the subject does not elicit a neutralizing immune response against the rAAV. In some embodiments, the isolated nucleic acid comprises AAV inverted terminal repeats (ITRs) flanking the transgene. In some embodiments, the nucleic acid sequence encoding the one or more gene products is operably linked to a promoter. In some embodiments, the one or more gene products comprise a protein or an inhibitory nucleic acid.

In some embodiments, the AAV capsid protein has the sequence set forth in any one of SEQ ID NOs: 124, 149, 161, 175, or 358. In some embodiments, the transgene is expressed in the joint at elevated levels relative to a control, optionally wherein the control is an rAAV2 comprising a wild-type AAV2 capsid protein.

In some embodiments, the one of more gene products is an anti-inflammatory gene product, optionally wherein the anti-inflammatory gene product is selected from the group consisting of: Insulin-like growth factor 1 (IGF-1), IL-Ira, IL-4, IL-6, IL-10, IL-11, IL-13, TGF-3, TNF receptor p55 (sTNFRI or sTNFRp55), TNF receptor p75 (sTNFRII or sTNFRP75), IL-1 receptor type 2 (sIL-1RII), membrane-bound IL-1 receptor type 2 (mIL-1RII), and IL-18 binding protein (IL-18BP). In some embodiments, the one of more gene products inhibit the expression or function of a pro-inflammatory gene, optionally wherein the pro-inflammatory gene is IL-1, tumor necrosis factor (TNF), IL-12, IL-18, interferon gamma (IFNγ), and granulocyte-macrophage colony stimulating factor (GM-CSF).

Some aspects of the disclosure provide a method of reducing inflammation in a joint of a subject. In some embodiments, a method of reducing inflammation in a joint of a subject comprises administering to the subject a recombinant adeno-associated virus (rAAV) comprising: (i) an isolated nucleic acid comprising a transgene encoding one or more gene products of interest; and (ii) an adeno-associated acid (AAV) capsid protein having the sequence set forth in SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84, wherein the administration results in reduction of inflammation in the joint.

In some embodiments, the administration comprises intra-articular injection. In some embodiments, the joint is a knee joint, shoulder joint, elbow joint, hip joint, ankle joint, spinal joint, finger joint, or toe joint. In some embodiments, the joint comprises skin, muscle, bone cells, articular cartilage, meniscus, synovium, and/or ligament tissues.

In some embodiments, the subject is a mammal, optionally wherein the mammal is a human. In some embodiments, the subject is characterized by production of anti-AAV2 antibodies (e.g., neutralizing antibodies (NAbs) against AAV2) prior to the administration. In some embodiments, after administration of the rAAV, the subject does not elicit a neutralizing immune response against the rAAV. In some embodiments, the isolated nucleic acid comprises AAV inverted terminal repeats (ITRs) flanking the transgene. In some embodiments, the nucleic acid sequence encoding the one or more gene products is operably linked to a promoter. In some embodiments, the one or more gene products comprise a protein or an inhibitory nucleic acid.

In some embodiments, the AAV capsid protein has the sequence set forth in any one of SEQ ID NOs: 124, 149, 161, 175, or 358. In some embodiments, the transgene is expressed in the joint at elevated levels relative to a control, optionally wherein the control is an rAAV2 comprising a wild-type AAV2 capsid protein.

In some embodiments, the one of more gene products is an anti-inflammatory gene product, optionally wherein the anti-inflammatory gene product is selected from the group consisting of: Insulin-like growth factor 1 (IGF-1), IL-Ira, IL-4, IL-6, IL-10, IL-11, IL-13, TGF-β, TNF receptor p55 (sTNFRI or sTNFRp55), TNF receptor p75 (sTNFRII or sTNFRP75), IL-1 receptor type 2 (sIL-1RII), membrane-bound IL-1 receptor type 2 (mIL-1RII), and IL-18 binding protein (IL-18BP). In some embodiments, the one of more gene products inhibit the expression or function of a pro-inflammatory gene, optionally wherein the pro-inflammatory gene is IL-1, tumor necrosis factor (TNF), IL-12, IL-18, interferon gamma (IFNγ), and granulocyte-macrophage colony stimulating factor (GM-CSF).

In some embodiments, the level of inflammation in the joint is reduced by at least 10%, at least 20%, at least 25%, at least 50%, at least 75%, or at least 100% relative to a control, optionally wherein the control is a joint that has been treated with an rAAV2 comprising a wild-type AAV2 capsid protein or the control is an untreated joint.

In some embodiments, the subject has osteoarthritis (OA) or rheumatoid arthritis (RA) in the joint.

### BRIEF DESCRIPTION OF DRAWINGS

FIG. 1 shows that AAV2 transduces mouse knee joints. A mouse knee joint was harvested 1 week after intra-articular injection of an AAV2 vector encoding enhanced green fluorescent protein (EGFP). The presence of EGFP was observed using a fluorescence microscope in the joint space (AC: articular cartilage; M: meniscus). A mouse knee joint that has not been injected with an AAV2 vector encoding EGFP is also shown.

FIG. 2 shows the disorganization and cell death of articular cartilage observed two weeks after surgical destabilization of the medial meniscus (DMM), a procedure that mimics the clinical progression after a meniscal injury as a model of osteoarthritis (OA).

FIG. 3 shows a high-throughput sequencing method for evaluating AAV2 capsid variants for knee joint tropism.

FIG. 4 shows a bioluminescence image of firefly luciferase in an OA model knee joint (circled) and a normal knee joint (not circled) of a mouse two weeks after intra-articular injection of a mixture of AAVs having unique capsid variants and expressing firefly luciferase.

FIGS. 5A-5C show sequencing results from the high-throughput sequencing method for evaluating AAV2 capsid variants for knee joint tropism. Forty-two AAV capsids were tested in an OA model knee joint in mice.

FIGS. 6A-6C show sequencing results from the high-throughput sequencing method for evaluating AAV2 capsid variants. Forty-two AAV capsids were tested in normal knee joints in mice.

FIG. 7 shows a graph illustrating that unique capsid variants (v149, v161, and v175) are capable of transducing knee joints for transgene delivery.

### DETAILED DESCRIPTION

Aspects of the disclosure relate to compositions and methods for delivering a transgene (e.g., an anti-inflammatory transgene encoding one or more gene products) to a joint (e.g., an arthritic joint). The disclosure is based, in part, on adeno-associated virus (AAV) capsid protein variants characterized by tropisms for certain cell types present in joints (e.g., cartilage, skin, muscle, and bone cells of a joint). In some embodiments, recombinant AAVs (rAAVs) comprising the capsid protein variants are more efficiently packaged than rAAVs having certain wild-type AAV capsid proteins. Methods of delivering an rAAV comprising the AAV capsid protein variants and methods of treating arthritis (e.g., osteoarthritis) are also described by the disclosure.

In some aspects, the disclosure provides methods for delivering a transgene (e.g., an anti-inflammatory transgene or a transgene that inhibits a pro-inflammatory gene) to a joint in a subject, the method comprising administering (e.g., by intra-articular injection) to the subject a recombinant adeno-associated virus (rAAV) comprising an isolated nucleic acid comprising a transgene encoding one or more gene products of interest; and an adeno-associated acid (AAV) capsid protein having the sequence set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84. In some embodiments, an adeno-associated acid (AAV) capsid protein for delivering a transgene (e.g., an anti-inflammatory transgene or a transgene that inhibits a pro-inflammatory gene) to a joint in a subject has the sequence set forth in any one of SEQ ID NOs: 124, 149, 161, 175, or 358. In some embodiments, an adeno-associated acid (AAV) capsid protein for delivering a transgene (e.g., an anti-inflammatory transgene or a transgene that inhibits a pro-inflammatory gene) to a joint in a subject is AAVv149, AAVv124, AAVv358, AAVv161, or AAVv175. In some embodiments, a capsid protein described herein (e.g., capsid protein having the sequence set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84) or a capsid protein having substantial homology to a capsid protein described herein (e.g., capsid protein having the sequence set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84) comprises one or more mutations in the Variable Region (VR)-IV, VR-V, VR-VI, VT-VII, and/or VR-VIII, relative to AAV2.

“Homology” refers to the percent identity between two polynucleotide or two polypeptide moieties. The term “substantial homology”, when referring to a nucleic acid, or fragment thereof, indicates that, when optimally aligned with appropriate nucleotide insertions or deletions with another nucleic acid (or its complementary strand), there is nucleotide sequence identity in about 90 to 100% of the aligned sequences. When referring to a polypeptide, or fragment thereof, the term “substantial homology” indicates that, when optimally aligned with appropriate gaps, insertions or deletions with another polypeptide, there is nucleotide sequence identity in about 90 to 100% of the aligned sequences. The term “highly conserved” means at least 80% identity, preferably at least 90% identity, and more preferably, over 97% identity. In some cases, highly conserved may refer to 100% identity. Identity is readily determined by one of skill in the art by, for example, the use of algorithms and computer programs known by those of skill in the art.

As described herein, alignments between sequences of nucleic acids or polypeptides are performed using any of a variety of publicly or commercially available Multiple Sequence Alignment Programs, such as “Clustal W”, accessible through Web Servers on the internet. Alternatively, Vector NTI utilities may also be used. There are also a number of algorithms known in the art that can be used to measure nucleotide sequence identity, including those contained in the programs described above. As another example, polynucleotide sequences can be compared using BLASTN, which provides alignments and percent sequence identity of the regions of the best overlap between the query and search sequences. Similar programs are available for the comparison of amino acid sequences, e.g., the “Clustal X” program, BLASTP. Typically, any of these programs are used at default settings, although one of skill in the art can alter these settings as needed. Alternatively, one of skill in the art can utilize another algorithm or computer program that provides at least the level of identity or alignment as that provided by the referenced algorithms and programs. Alignments may be used to identify corresponding amino acids between two proteins or peptides. A “corresponding amino acid” is an amino acid of a protein or peptide sequence that has been aligned with an amino acid of another protein or peptide sequence. Corresponding amino acids may be identical or non-identical. A corresponding amino acid that is a non-identical amino acid may be referred to as a variant amino acid.

In some aspects, the disclosure relates to a capsid protein (e.g., an isolated nucleic acid encoding a capsid protein having the sequence set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84; a recombinant adeno-associated virus (rAAV) comprising a capsid protein having the sequence set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84; etc.), or a capsid protein having substantial homology to a capsid protein described herein. In some embodiments, a capsid protein having substantial homology to a capsid protein having the sequence set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84 is at least 50%, 60%, 70%, 80%, 90%, 95%, or 99% identical to the amino acid sequence set forth in SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84. In some embodiments, a capsid protein having substantial homology to a capsid protein having the sequence set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84 comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions, insertions, or deletions, relative to the base amino acid sequence. In some embodiments, a capsid protein having substantial homology comprises more than 50 amino acid substitutions, insertions, or deletions, relative to the base amino acid sequence.

The disclosure relates, in some aspects, to the surprising discovery that rAAVs comprising capsid protein having the sequence set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84 are able to be produced in higher quantities in mammalian cell lines (e.g., HEK-293 cells) relative to rAAVs having certain other AAV capsid proteins (e.g., AAV2 capsid proteins, AAV3B capsid proteins, etc.). In some embodiments, transduced mammalian (e.g., HEK) producer cells yield between about 1.5-fold and about 5-fold (e.g., 1.5, 2, 3, 4, 5-fold) more rAAVs having capsid protein having the sequence set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84 than mammalian (e.g., HEK) producer cells transduced with AAV2 capsid proteins. In some embodiments, transduced mammalian (e.g., HEK) producer cells yield between about 5% and about 50% (e.g., 5%, 10%, 15%, 20%, 25%, 30%, 35%, 40%, 45%, 50%, etc.) more rAAVs having capsid protein having the sequence set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84 than mammalian (e.g., HEK) producer cells transduced with AAV3B capsid proteins.

Aspects of the disclosure relate to the unexpectedly improved transduction efficiency into a joint (e.g., skin, muscle, bone cells, articular cartilage, meniscus, synovium, and/or ligament tissues) of a subject by a capsid protein having the sequence set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84 (e.g., rAAVs comprising a capsid protein having the sequence set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84) relative to rAAVs having AAV2 capsid proteins. In some embodiments, such rAAVs of the disclosure transduce a joint at least 5%, 10%, 15%, 20%, 25%, 30%, 40%, 50%, 100%, 200%, 500%, 1000%, or more efficiently than AAV2-containing rAAVs. In some embodiments, the joint comprises skin, muscle, bone cells, articular cartilage, meniscus, synovium, and/or ligament tissues.

Aspects of the disclosure relate to certain AAV capsid proteins (e.g., a capsid protein having the sequence set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84) that are serologically distinct from other AAV capsid proteins (e.g., AAV1, AAV2, AAV3B, AAV8, AAV9, AAVrh.8, AAVrh.10, etc.). Without wishing to be bound by any particular theory, rAAVs comprising the variant capsid proteins of the disclosure are not subject to the neutralizing antibody response in a subject that is sero-positive for antibodies against certain other AAV capsids. For example, rAAVs comprising a capsid protein having the sequence set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84 are not subject to neutralizing antibodies (NAbs) against AAV2. Accordingly, in some embodiments, rAAVs comprising the variant capsid proteins of the disclosure may be useful as a second-line therapy for delivery of transgenes to subjects that have previously been administered AAV therapies, or that are sero-positive for certain AAV capsid neutralizing antibodies (e.g., NAbs against AAV2).

In some aspects, the disclosure relates to rAAV variant capsid proteins that exhibit increased thermostability relative to certain wild-type AAV capsid proteins (e.g., AAV2 capsid protein). In some embodiments, a variant capsid protein is more thermostable than an AAV2 capsid protein at a pH ranging from about pH 4 to about pH 7. In some embodiments, thermostability is determined by calculating the melting temperature of a capsid protein. In some embodiments, a variant capsid protein is characterized by a melting temperature that is between about 5° C. and about 10° C. above the melting temperature of an AAV2 capsid protein, at a given pH (e.g., between pH 4 and pH 7).

### Isolated Nucleic Acids

In some aspects, the disclosure relates to isolated nucleic acids encoding certain AAV capsid protein variants (e.g., a capsid protein having the sequence set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84). A “nucleic acid” sequence refers to a DNA or RNA sequence. In some embodiments, the term nucleic acid captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

In some embodiments, proteins and nucleic acids of the disclosure are isolated. As used herein, the term “isolated” means artificially obtained or produced. As used herein with respect to nucleic acids, the term “isolated” generally means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one that is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” generally refers to a protein or peptide that has been artificially obtained or produced (e.g., by chemical synthesis, by recombinant DNA technology, etc.).

It should be appreciated that conservative amino acid substitutions may be made to provide functionally equivalent variants, or homologs of the capsid proteins. In some aspects the disclosure embraces sequence alterations that result in conservative amino acid substitutions. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the proteins and polypeptides disclosed herein.

Recombinant AAVs (rAAVs)

In some aspects, the disclosure provides isolated AAVs. As used herein with respect to AAVs, the term “isolated” refers to an AAV that has been artificially obtained or produced. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected. In some embodiments, the rAAV comprises an AAVv66 capsid protein. In some embodiments, the rAAV comprises a capsid protein having an amino acid sequence as set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84.

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV variant capsid protein or fragment thereof; a functional rep gene; a recombinant AAV vector composed of, AAV inverted terminal repeats (ITRs) and a transgene; and sufficient helper functions to permit packaging of the recombinant AAV vector into the AAV capsid proteins. In some embodiments, capsid proteins are structural proteins encoded by a cap gene of an AAV. In some embodiments, AAVs comprise three capsid proteins, virion proteins 1 to 3 (named VP1, VP2 and VP3), all of which may be expressed from a single cap gene. Accordingly, in some embodiments, the VP1, VP2 and VP3 proteins share a common core sequence. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the protein shell is primarily comprised of a VP3 capsid protein. In some embodiments, the functions of the capsid proteins are to protect the viral genome, deliver the genome and interact with the host. In some aspects, capsid proteins deliver the viral genome to a host in a tissue specific manner. In some embodiments, VP1 and/or VP2 capsid proteins may contribute to the tissue tropism of the packaged AAV. In some embodiments, the tissue tropism of the packaged AAV is determined by the VP3 capsid protein. In some embodiments, the tissue tropism of an AAV is enhanced or changed by mutations occurring in the capsid proteins.

In some aspects, rAAV variants described herein (e.g., comprising a capsid protein having an amino acid sequence as set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84) may be useful for reducing inflammation in a joint of a subject (e.g., by intra-articular injection of the rAAV to the subject). In some embodiments, the level of inflammation in the joint is reduced by at least 5%, 10%, 15%, 20%, 25%, 30%, 40%, 50%, 60%, 70%, 80%, 90%, 100%, 150%, or 150% relative to a control. In some embodiments, the level of inflammation in the joint is reduced by at least 5%, 10%, 15%, 20%, 25%, 30%, 40%, 50%, 60%, 70%, 80%, 90%, 100%, 150%, or 150% relative to a joint that has been treated with an rAAV2 comprising a wild-type AAV2 capsid protein. In some embodiments, the level of inflammation in the joint is reduced by at least 5%, 10%, 15%, 20%, 25%, 30%, 40%, 50%, 60%, 70%, 80%, 90%, 100%, 150%, or 150% relative to an untreated joint. In some embodiments, the joint has arthritis. In some embodiments, the arthritis is osteoarthritis (OA) or rheumatoid arthritis (RA).

In some embodiments, rAAV variants described herein (e.g., comprising a capsid protein having an amino acid sequence as set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84) may be useful for delivering a transgene to a joint of a subject (e.g., by intra-articular injection of the rAAV to the subject). In some embodiments, a transgene encodes an anti-inflammatory gene product. An anti-inflammatory gene product may be Insulin-like growth factor 1 (IGF-1), IL-Ira, IL-4, IL-6, IL-10, IL-11, IL-13, TGF-β, TNF receptor p55 (sTNFRI or sTNFRp55), TNF receptor p75 (sTNFRII or sTNFRP75), IL-1 receptor type 2 (sIL-1RII), membrane-bound IL-1 receptor type 2 (mIL-1RII), or IL-18 binding protein (IL-18BP). In some embodiments, a transgene encodes a gene product that inhibits a pro-inflammatory gene. In some embodiments, a gene product that inhibits a pro-inflammatory gene is a protein (e.g., a protein that inhibits transcription or translation of a pro-inflammatory gene; or a protein that binds to a pro-inflammatory gene product to inhibit its function). In some embodiments, a gene product that inhibits a pro-inflammatory gene is an inhibitory nucleic acid that inhibits transcription or translation of a pro-inflammatory gene. A pro-inflammatory gene product may be IL-1, tumor necrosis factor (TNF), IL-12, IL-18, interferon gamma (IFNγ), or granulocyte-macrophage colony stimulating factor (GM-CSF).

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions required for producing the rAAV of the disclosure may be delivered to the packaging host cell using any appropriate genetic element (vector). In some embodiments, a single nucleic acid encoding all three capsid proteins (e.g., VP1, VP2 and VP3) is delivered into the packaging host cell in a single vector. In some embodiments, nucleic acids encoding the capsid proteins are delivered into the packaging host cell by two vectors; a first vector comprising a first nucleic acid encoding two capsid proteins (e.g., VP1 and VP2) and a second vector comprising a second nucleic acid encoding a single capsid protein (e.g., VP3). In some embodiments, three vectors, each comprising a nucleic acid encoding a different capsid protein, are delivered to the packaging host cell. The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell (e.g., across the cell membrane). A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell that has been transfected. Thus, a “host cell” as used herein may refer to a cell that has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

Cells may also be transfected with a vector (e.g., helper vector) that provides helper functions to the AAV. The vector providing helper functions may provide adenovirus functions, including, e.g., E1a, E1b, E2a, and E4ORF6. The sequences of adenovirus gene providing these functions may be obtained from any known adenovirus serotype, such as serotypes 2, 3, 4, 7, 12 and 40, and further including any of the presently identified human types known in the art. Thus, in some embodiments, the methods involve transfecting the cell with a vector expressing one or more genes necessary for AAV replication, AAV gene transcription, and/or AAV packaging.

As used herein, the term “vector” includes any genetic element, such as a plasmid, phage, transposon, cosmid, chromosome, artificial chromosome, virus, virion, etc., that is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment (e.g., nucleic acid sequence) to be transcribed is positioned under the transcriptional control of a promoter. A “promoter” refers to a DNA sequence recognized by the synthetic machinery of the cell, or introduced synthetic machinery, that is required to initiate the specific transcription of a gene. The phrases “operatively positioned,” “under control” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or inhibitory RNA (e.g., shRNA, miRNA, miRNA inhibitor) from a transcribed gene.

In some embodiments, a promoter is a Cytomegalovirus early enhancer/chicken R actin (CB6) promoter.

In some cases, an isolated capsid gene can be used to construct and package recombinant AAVs, using methods well known in the art, to determine functional characteristics associated with the capsid protein encoded by the gene. For example, isolated capsid genes can be used to construct and package a recombinant AAV (rAAV) comprising a reporter gene (e.g., B-Galactosidase, GFP, Luciferase, etc.). The rAAV can then be delivered to an animal (e.g., mouse) and the tissue targeting properties of the novel isolated capsid gene can be determined by examining the expression of the reporter gene in various tissues (e.g., heart, liver, kidneys) of the animal. Other methods for characterizing the novel isolated capsid genes are disclosed herein and still others are well known in the art.

The foregoing methods for packaging recombinant vectors in desired AAV capsids to produce the rAAVs of the disclosure are not meant to be limiting and other suitable methods will be apparent to the skilled artisan.

rAAV Vectors

“Recombinant AAV (rAAV) vectors” of the disclosure are typically composed of, at a minimum, a transgene and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, that encodes a polypeptide, protein, functional RNA molecule (e.g., miRNA, miRNA inhibitor) or other gene product, of interest. The nucleic acid coding sequence is operatively linked to regulatory components in a manner that permits transgene transcription, translation, and/or expression in a cell of a target tissue.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). An example of such a molecule employed in the present disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types.

In some embodiments, the disclosure provides a self-complementary AAV vector. As used herein, the term “self-complementary AAV vector” (scAAV) refers to a vector containing a double-stranded vector genome generated by the absence of a terminal resolution site (TR) from one of the ITRs of the AAV. The absence of a TR prevents the initiation of replication at the vector terminus where the TR is not present. In general, scAAV vectors generate single-stranded, inverted repeat genomes, with a wild-type (wt) AAV TR at each end and a mutated TR (mTR) in the middle.

In some embodiments, the rAAVs of the present disclosure are pseudotyped rAAVs. Pseudotyping is the process of producing viruses or viral vectors in combination with foreign viral envelope proteins. The result is a pseudotyped virus particle. With this method, the foreign viral envelope proteins can be used to alter host tropism or an increased/decreased stability of the virus particles. In some aspects, a pseudotyped rAAV comprises nucleic acids from two or more different AAVs, wherein the nucleic acid from one AAV encodes a capsid protein and the nucleic acid of at least one other AAV encodes other viral proteins and/or the viral genome. In some embodiments, a pseudotyped rAAV refers to an AAV comprising an inverted terminal repeat (ITR) of one AAV serotype and a capsid protein of a different AAV serotype. For example, a pseudotyped AAV vector containing the ITRs of serotype X encapsidated with the proteins of Y will be designated as AAVX/Y (e.g., AAV2/1 has the ITRs of AAV2 and the capsid of AAV1). In some embodiments, pseudotyped rAAVs may be useful for combining the tissue-specific targeting capabilities of a capsid protein from one AAV serotype with the viral DNA from another AAV serotype, thereby allowing targeted delivery of a transgene to a target tissue.

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus produced by the disclosure. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A great number of expression control sequences, including promoters that are native, constitutive, inducible and/or tissue-specific, are known in the art and may be utilized.

As used herein, a nucleic acid sequence (e.g., coding sequence) and regulatory sequences are said to be “operably” linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., shRNA, miRNA, miRNA inhibitor).

For nucleic acids encoding proteins, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contains more than one polypeptide chains. Selection of these and other common vector elements are conventional and many such sequences are available [see, e.g., Sambrook et al, and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, P et al., Human Gene Therapy, 2000; 11: 1921-1931.; and Klump, H et al., Gene Therapy, 2001; 8: 811-817).

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors of the disclosure may optionally include 5′ leader or signal sequences. The choice and design of an appropriate vector is within the ability and discretion of one of ordinary skill in the art.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the 3-actin promoter, the phosphoglycerol kinase (PGK) promoter, and the EF1α promoter [Invitrogen].

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, Proc. Natl. Acad. Sci. USA, 93:3346-3351 (1996)), the tetracycline-repressible system (Gossen et al, Proc. Natl. Acad. Sci. USA, 89:5547-5551 (1992)), the tetracycline-inducible system (Gossen et al, Science, 268:1766-1769 (1995), see also Harvey et al, Curr. Opin. Chem. Biol., 2:512-518 (1998)), the RU486-inducible system (Wang et al, Nat. Biotech., 15:239-243 (1997) and Wang et al, Gene Ther., 4:432-441 (1997)) and the rapamycin-inducible system (Magari et al, J. Clin. Invest., 100:2865-2872 (1997)). Still other types of inducible promoters that may be useful in this context are those that are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, a gastrointestinal-specific mucin-2 promoter, an eye-specific retinoschisin promoter, an eye-specific K12 promoter, a respiratory tissue-specific CC10 promoter, a respiratory tissue-specific surfactant protein C (SP-C) promoter, a breast tissue-specific PRC1 promoter, a breast tissue-specific RRM2 promoter, a urinary tract tissue-specific uroplakin 2 (UPII) promoter, a uterine tissue-specific lactoferrin promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan. In some embodiments, a tissue-specific regulatory sequence is a promoter that is specific for cartilage, skin, muscle, or bone cells.

In some embodiments, one or more bindings sites for one or more of miRNAs are incorporated in a transgene of a rAAV vector, to inhibit the expression of the transgene in one or more tissues of a subject harboring the transgene (e.g, detargeting of transgene expression in a cell-type specific manner). The skilled artisan will appreciate that binding sites may be selected to control the expression of a transgene in a tissue specific manner. For example, binding sites for the liver-specific miR-122 may be incorporated into a transgene to inhibit expression of that transgene in the liver. The target sites in the mRNA may be in the 5′ UTR, the 3′ UTR or in the coding region. Typically, the target site is in the 3′ UTR of the mRNA. Furthermore, the transgene may be designed such that multiple miRNAs regulate the mRNA by recognizing the same or multiple sites. The presence of multiple miRNA binding sites may result in the cooperative action of multiple RISCs and provide highly efficient inhibition of expression. The target site sequence may comprise a total of 5-100, 10-60, or more nucleotides. The target site sequence may comprise at least 5 nucleotides of the sequence of a target gene binding site.

In some embodiments, a transgene comprises one or more (e.g., 1, 2, 3, 4, 5, or more) miRNA binding sites that de-target expression of the transgene from immune cells (e.g., antigen presenting cells (APCs), such as macrophages, dendrites, etc.). Incorporation of miRNA binding sites for immune-associated miRNAs may de-target transgene expression from antigen presenting cells and thus reduce or eliminate immune responses (cellular and/or humoral) produced in the subject against products of the transgene, for example as described in US 2018/0066279, the entire contents of which are incorporated herein by reference. In some embodiments, the immune-associated miRNA is selected from: miR-15a, miR-16-1, miR-17, miR-18a, miR-19a, miR-19b-1, miR-20a, miR-21, miR-29a/b/c, miR-30b, miR-31, miR-34a, miR-92a-1, miR-106a, miR-125a/b, miR-142-3p, miR-146a, miR-150, miR-155, miR-181a, miR-223 and miR-424, miR-221, miR-222, let-7i, miR-148, and miR-152.

The composition of the transgene sequence of the rAAV vector will depend upon the use to which the resulting vector will be put. For example, one type of transgene sequence includes a reporter sequence, which upon expression produces a detectable signal. In another example, the transgene encodes a therapeutic protein or therapeutic functional RNA. In another example, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. Appropriate transgene coding sequences will be apparent to the skilled artisan.

Reporter sequences that may be provided in a transgene include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer. Such reporters can, for example, be useful in verifying the tissue-specific targeting capabilities and tissue specific promoter regulatory activity of an rAAV.

In some aspects, the disclosure provides rAAV vectors for use in methods of preventing or treating one or more genetic deficiencies or dysfunctions in a mammal, such as for example, a polypeptide deficiency or polypeptide excess in a mammal, and particularly for treating or reducing the severity or extent of deficiency in a human manifesting one or more of the disorders linked to a deficiency in such polypeptides in cells and tissues. The method involves administration of an rAAV vector that encodes one or more therapeutic peptides, polypeptides, siRNAs, microRNAs, antisense nucleotides, etc. in a pharmaceutically-acceptable carrier to the subject in an amount and for a period of time sufficient to treat the deficiency or disorder in the subject suffering from such a disorder.

Thus, the disclosure embraces the delivery of rAAV vectors encoding one or more peptides, polypeptides, or proteins, which are useful for the treatment or prevention of disease states in a mammalian subject. Exemplary therapeutic proteins include one or more polypeptides selected from the group consisting of growth factors, interleukins, interferons, anti-apoptosis factors, cytokines, anti-diabetic factors, anti-apoptosis agents, coagulation factors, anti-tumor factors. Other non-limiting examples of therapeutic proteins include BDNF, CNTF, CSF, EGF, FGF, G-SCF, GM-CSF, gonadotropin, IFN, IFG-1, M-CSF, NGF, PDGF, PEDF, TGF, VEGF, TGF-B2, TNF, prolactin, somatotropin, XIAP1, IL-1, IL-2, IL-3, IL-4, IL-5, IL-6, IL-7, IL-8, IL-9, IL-10, IL-10 (187A), viral IL-10, IL-11, IL-12, IL-13, IL-14, IL-15, IL-16 IL-17, and IL-18. The rAAV vectors may comprise a gene to be transferred to a subject to treat a disease associated with reduced expression, lack of expression or dysfunction of the gene.

The skilled artisan will also realize that in the case of transgenes encoding proteins or polypeptides, that mutations that results in conservative amino acid substitutions may be made in a transgene to provide functionally equivalent variants, or homologs of a protein or polypeptide. In some aspects the disclosure embraces sequence alterations that result in conservative amino acid substitution of a transgene. In some embodiments, the transgene comprises a gene having a dominant negative mutation. For example, a transgene may express a mutant protein that interacts with the same elements as a wild-type protein, and thereby blocks some aspect of the function of the wild-type protein.

Useful transgene products also include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors that are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA.

The following non-limiting list of miRNA genes, and their homologues, are useful as transgenes or as targets for small interfering nucleic acids encoded by transgenes (e.g., miRNA sponges, antisense oligonucleotides, TuD RNAs) in certain embodiments of the methods: hsa-let-7a, hsa-let-7a*, hsa-let-7b, hsa-let-7b*, hsa-let-7c, hsa-let-7c*, hsa-let-7d, hsa-let-7d*, hsa-let-7e, hsa-let-7e*, hsa-let-7f, hsa-let-7f-1*, hsa-let-7f-2*, hsa-let-7g, hsa-let-7g*, hsa-let-7i, hsa-let-7i*, hsa-miR-1, hsa-miR-100, hsa-miR-100*, hsa-miR-101, hsa-miR-101*, hsa-miR-103, hsa-miR-105, hsa-miR-105*, hsa-miR-106a, hsa-miR-106a*, hsa-miR-106b, hsa-miR-106b*, hsa-miR-107, hsa-miR-10a, hsa-miR-10a*, hsa-miR-10b, hsa-miR-10b*, hsa-miR-1178, hsa-miR-1179, hsa-miR-1180, hsa-miR-1181, hsa-miR-1182, hsa-miR-1183, hsa-miR-1184, hsa-miR-1185, hsa-miR-1197, hsa-miR-1200, hsa-miR-1201, hsa-miR-1202, hsa-miR-1203, hsa-miR-1204, hsa-miR-1205, hsa-miR-1206, hsa-miR-1207-3p, hsa-miR-1207-5p, hsa-miR-1208, hsa-miR-122, hsa-miR-122*, hsa-miR-1224-3p, hsa-miR-1224-5p, hsa-miR-1225-3p, hsa-miR-1225-5p, hsa-miR-1226, hsa-miR-1226*, hsa-miR-1227, hsa-miR-1228, hsa-miR-1228*, hsa-miR-1229, hsa-miR-1231, hsa-miR-1233, hsa-miR-1234, hsa-miR-1236, hsa-miR-1237, hsa-miR-1238, hsa-miR-124, hsa-miR-124*, hsa-miR-1243, hsa-miR-1244, hsa-miR-1245, hsa-miR-1246, hsa-miR-1247, hsa-miR-1248, hsa-miR-1249, hsa-miR-1250, hsa-miR-1251, hsa-miR-1252, hsa-miR-1253, hsa-miR-1254, hsa-miR-1255a, hsa-miR-1255b, hsa-miR-1256, hsa-miR-1257, hsa-miR-1258, hsa-miR-1259, hsa-miR-125a-3p, hsa-miR-125a-5p, hsa-miR-125b, hsa-miR-125b-1*, hsa-miR-125b-2*, hsa-miR-126, hsa-miR-126*, hsa-miR-1260, hsa-miR-1261, hsa-miR-1262, hsa-miR-1263, hsa-miR-1264, hsa-miR-1265, hsa-miR-1266, hsa-miR-1267, hsa-miR-1268, hsa-miR-1269, hsa-miR-1270, hsa-miR-1271, hsa-miR-1272, hsa-miR-1273, hsa-miR-127-3p, hsa-miR-1274a, hsa-miR-1274b, hsa-miR-1275, hsa-miR-127-5p, hsa-miR-1276, hsa-miR-1277, hsa-miR-1278, hsa-miR-1279, hsa-miR-128, hsa-miR-1280, hsa-miR-1281, hsa-miR-1282, hsa-miR-1283, hsa-miR-1284, hsa-miR-1285, hsa-miR-1286, hsa-miR-1287, hsa-miR-1288, hsa-miR-1289, hsa-miR-129*, hsa-miR-1290, hsa-miR-1291, hsa-miR-1292, hsa-miR-1293, hsa-miR-129-3p, hsa-miR-1294, hsa-miR-1295, hsa-miR-129-5p, hsa-miR-1296, hsa-miR-1297, hsa-miR-1298, hsa-miR-1299, hsa-miR-1300, hsa-miR-1301, hsa-miR-1302, hsa-miR-1303, hsa-miR-1304, hsa-miR-1305, hsa-miR-1306, hsa-miR-1307, hsa-miR-1308, hsa-miR-130a, hsa-miR-130a*, hsa-miR-130b, hsa-miR-130b*, hsa-miR-132, hsa-miR-132*, hsa-miR-1321, hsa-miR-1322, hsa-miR-1323, hsa-miR-1324, hsa-miR-133a, hsa-miR-133b, hsa-miR-134, hsa-miR-135a, hsa-miR-135a*, hsa-miR-135b, hsa-miR-135b*, hsa-miR-136, hsa-miR-136*, hsa-miR-137, hsa-miR-138, hsa-miR-138-1*, hsa-miR-138-2*, hsa-miR-139-3p, hsa-miR-139-5p, hsa-miR-140-3p, hsa-miR-140-5p, hsa-miR-141, hsa-miR-141*, hsa-miR-142-3p, hsa-miR-142-5p, hsa-miR-143, hsa-miR-143*, hsa-miR-144, hsa-miR-144*, hsa-miR-145, hsa-miR-145*, hsa-miR-146a, hsa-miR-146a*, hsa-miR-146b-3p, hsa-miR-146b-5p, hsa-miR-147, hsa-miR-147b, hsa-miR-148a, hsa-miR-148a*, hsa-miR-148b, hsa-miR-148b*, hsa-miR-149, hsa-miR-149*, hsa-miR-150, hsa-miR-150*, hsa-miR-151-3p, hsa-miR-151-5p, hsa-miR-152, hsa-miR-153, hsa-miR-154, hsa-miR-154*, hsa-miR-155, hsa-miR-155*, hsa-miR-15a, hsa-miR-15a*, hsa-miR-15b, hsa-miR-15b*, hsa-miR-16, hsa-miR-16-1*, hsa-miR-16-2*, hsa-miR-17, hsa-miR-17*, hsa-miR-181a, hsa-miR-181a*, hsa-miR-181a-2*, hsa-miR-181b, hsa-miR-181c, hsa-miR-181c*, hsa-miR-181d, hsa-miR-182, hsa-miR-182*, hsa-miR-1825, hsa-miR-1826, hsa-miR-1827, hsa-miR-183, hsa-miR-183*, hsa-miR-184, hsa-miR-185, hsa-miR-185*, hsa-miR-186, hsa-miR-186*, hsa-miR-187, hsa-miR-187*, hsa-miR-188-3p, hsa-miR-188-5p, hsa-miR-18a, hsa-miR-18a*, hsa-miR-18b, hsa-miR-18b*, hsa-miR-190, hsa-miR-190b, hsa-miR-191, hsa-miR-191*, hsa-miR-192, hsa-miR-192*, hsa-miR-193a-3p, hsa-miR-193a-5p, hsa-miR-193b, hsa-miR-193b*, hsa-miR-194, hsa-miR-194*, hsa-miR-195, hsa-miR-195*, hsa-miR-196a, hsa-miR-196a*, hsa-miR-196b, hsa-miR-197, hsa-miR-198, hsa-miR-199a-3p, hsa-miR-199a-5p, hsa-miR-199b-5p, hsa-miR-19a, hsa-miR-19a*, hsa-miR-19b, hsa-miR-19b-1*, hsa-miR-19b-2*, hsa-miR-200a, hsa-miR-200a*, hsa-miR-200b, hsa-miR-200b*, hsa-miR-200c, hsa-miR-200c*, hsa-miR-202, hsa-miR-202*, hsa-miR-203, hsa-miR-204, hsa-miR-205, hsa-miR-206, hsa-miR-208a, hsa-miR-208b, hsa-miR-20a, hsa-miR-20a*, hsa-miR-20b, hsa-miR-20b*, hsa-miR-21, hsa-miR-21*, hsa-miR-210, hsa-miR-211, hsa-miR-212, hsa-miR-214, hsa-miR-214*, hsa-miR-215, hsa-miR-216a, hsa-miR-216b, hsa-miR-217, hsa-miR-218, hsa-miR-218-1*, hsa-miR-218-2*, hsa-miR-219-1-3p, hsa-miR-219-2-3p, hsa-miR-219-5p, hsa-miR-22, hsa-miR-22*, hsa-miR-220a, hsa-miR-220b, hsa-miR-220c, hsa-miR-221, hsa-miR-221*, hsa-miR-222, hsa-miR-222*, hsa-miR-223, hsa-miR-223*, hsa-miR-224, hsa-miR-23a, hsa-miR-23a*, hsa-miR-23b, hsa-miR-23b*, hsa-miR-24, hsa-miR-24-1*, hsa-miR-24-2*, hsa-miR-25, hsa-miR-25*, hsa-miR-26a, hsa-miR-26a-1*, hsa-miR-26a-2*, hsa-miR-26b, hsa-miR-26b*, hsa-miR-27a, hsa-miR-27a*, hsa-miR-27b, hsa-miR-27b*, hsa-miR-28-3p, hsa-miR-28-5p, hsa-miR-296-3p, hsa-miR-296-5p, hsa-miR-297, hsa-miR-298, hsa-miR-299-3p, hsa-miR-299-5p, hsa-miR-29a, hsa-miR-29a*, hsa-miR-29b, hsa-miR-29b-1*, hsa-miR-29b-2*, hsa-miR-29c, hsa-miR-29c*, hsa-miR-300, hsa-miR-301a, hsa-miR-301b, hsa-miR-302a, hsa-miR-302a*, hsa-miR-302b, hsa-miR-302b*, hsa-miR-302c, hsa-miR-302c*, hsa-miR-302d, hsa-miR-302d*, hsa-miR-302e, hsa-miR-302f, hsa-miR-30a, hsa-miR-30a*, hsa-miR-30b, hsa-miR-30b*, hsa-miR-30c, hsa-miR-30c-1*, hsa-miR-30c-2*, hsa-miR-30d, hsa-miR-30d*, hsa-miR-30e, hsa-miR-30e*, hsa-miR-31, hsa-miR-31*, hsa-miR-32, hsa-miR-32*, hsa-miR-320a, hsa-miR-320b, hsa-miR-320c, hsa-miR-320d, hsa-miR-323-3p, hsa-miR-323-5p, hsa-miR-324-3p, hsa-miR-324-5p, hsa-miR-325, hsa-miR-326, hsa-miR-328, hsa-miR-329, hsa-miR-330-3p, hsa-miR-330-5p, hsa-miR-331-3p, hsa-miR-331-5p, hsa-miR-335, hsa-miR-335*, hsa-miR-337-3p, hsa-miR-337-5p, hsa-miR-338-3p, hsa-miR-338-5p, hsa-miR-339-3p, hsa-miR-339-5p, hsa-miR-33a, hsa-miR-33a*, hsa-miR-33b, hsa-miR-33b*, hsa-miR-340, hsa-miR-340*, hsa-miR-342-3p, hsa-miR-342-5p, hsa-miR-345, hsa-miR-346, hsa-miR-34a, hsa-miR-34a*, hsa-miR-34b, hsa-miR-34b*, hsa-miR-34c-3p, hsa-miR-34c-5p, hsa-miR-361-3p, hsa-miR-361-5p, hsa-miR-362-3p, hsa-miR-362-5p, hsa-miR-363, hsa-miR-363*, hsa-miR-365, hsa-miR-367, hsa-miR-367*, hsa-miR-369-3p, hsa-miR-369-5p, hsa-miR-370, hsa-miR-371-3p, hsa-miR-371-5p, hsa-miR-372, hsa-miR-373, hsa-miR-373*, hsa-miR-374a, hsa-miR-374a*, hsa-miR-374b, hsa-miR-374b*, hsa-miR-375, hsa-miR-376a, hsa-miR-376a*, hsa-miR-376b, hsa-miR-376c, hsa-miR-377, hsa-miR-377*, hsa-miR-378, hsa-miR-378*, hsa-miR-379, hsa-miR-379*, hsa-miR-380, hsa-miR-380*, hsa-miR-381, hsa-miR-382, hsa-miR-383, hsa-miR-384, hsa-miR-409-3p, hsa-miR-409-5p, hsa-miR-410, hsa-miR-411, hsa-miR-411*, hsa-miR-412, hsa-miR-421, hsa-miR-422a, hsa-miR-423-3p, hsa-miR-423-5p, hsa-miR-424, hsa-miR-424*, hsa-miR-425, hsa-miR-425*, hsa-miR-429, hsa-miR-431, hsa-miR-431*, hsa-miR-432, hsa-miR-432*, hsa-miR-433, hsa-miR-448, hsa-miR-449a, hsa-miR-449b, hsa-miR-450a, hsa-miR-450b-3p, hsa-miR-450b-5p, hsa-miR-451, hsa-miR-452, hsa-miR-452*, hsa-miR-453, hsa-miR-454, hsa-miR-454*, hsa-miR-455-3p, hsa-miR-455-5p, hsa-miR-483-3p, hsa-miR-483-5p, hsa-miR-484, hsa-miR-485-3p, hsa-miR-485-5p, hsa-miR-486-3p, hsa-miR-486-5p, hsa-miR-487a, hsa-miR-487b, hsa-miR-488, hsa-miR-488*, hsa-miR-489, hsa-miR-490-3p, hsa-miR-490-5p, hsa-miR-491-3p, hsa-miR-491-5p, hsa-miR-492, hsa-miR-493, hsa-miR-493*, hsa-miR-494, hsa-miR-495, hsa-miR-496, hsa-miR-497, hsa-miR-497*, hsa-miR-498, hsa-miR-499-3p, hsa-miR-499-5p, hsa-miR-500, hsa-miR-500*, hsa-miR-501-3p, hsa-miR-501-5p, hsa-miR-502-3p, hsa-miR-502-5p, hsa-miR-503, hsa-miR-504, hsa-miR-505, hsa-miR-505*, hsa-miR-506, hsa-miR-507, hsa-miR-508-3p, hsa-miR-508-5p, hsa-miR-509-3-5p, hsa-miR-509-3p, hsa-miR-509-5p, hsa-miR-510, hsa-miR-511, hsa-miR-512-3p, hsa-miR-512-5p, hsa-miR-513a-3p, hsa-miR-513a-5p, hsa-miR-513b, hsa-miR-513c, hsa-miR-514, hsa-miR-515-3p, hsa-miR-515-5p, hsa-miR-516a-3p, hsa-miR-516a-5p, hsa-miR-516b, hsa-miR-517*, hsa-miR-517a, hsa-miR-517b, hsa-miR-517c, hsa-miR-518a-3p, hsa-miR-518a-5p, hsa-miR-518b, hsa-miR-518c, hsa-miR-518c*, hsa-miR-518d-3p, hsa-miR-518d-5p, hsa-miR-518e, hsa-miR-518e*, hsa-miR-518f, hsa-miR-518f*, hsa-miR-519a, hsa-miR-519b-3p, hsa-miR-519c-3p, hsa-miR-519d, hsa-miR-519e, hsa-miR-519e*, hsa-miR-520a-3p, hsa-miR-520a-5p, hsa-miR-520b, hsa-miR-520c-3p, hsa-miR-520d-3p, hsa-miR-520d-5p, hsa-miR-520e, hsa-miR-520f, hsa-miR-520g, hsa-miR-520h, hsa-miR-521, hsa-miR-522, hsa-miR-523, hsa-miR-524-3p, hsa-miR-524-5p, hsa-miR-525-3p, hsa-miR-525-5p, hsa-miR-526b, hsa-miR-526b*, hsa-miR-532-3p, hsa-miR-532-5p, hsa-miR-539, hsa-miR-541, hsa-miR-541*, hsa-miR-542-3p, hsa-miR-542-5p, hsa-miR-543, hsa-miR-544, hsa-miR-545, hsa-miR-545*, hsa-miR-548a-3p, hsa-miR-548a-5p, hsa-miR-548b-3p, hsa-miR-548b-5p, hsa-miR-548c-3p, hsa-miR-548c-5p, hsa-miR-548d-3p, hsa-miR-548d-5p, hsa-miR-548e, hsa-miR-548f, hsa-miR-548g, hsa-miR-548h, hsa-miR-548i, hsa-miR-548j, hsa-miR-548k, hsa-miR-548l, hsa-miR-548m, hsa-miR-548n, hsa-miR-548o, hsa-miR-548p, hsa-miR-549, hsa-miR-550, hsa-miR-550*, hsa-miR-551a, hsa-miR-551b, hsa-miR-551b*, hsa-miR-552, hsa-miR-553, hsa-miR-554, hsa-miR-555, hsa-miR-556-3p, hsa-miR-556-5p, hsa-miR-557, hsa-miR-558, hsa-miR-559, hsa-miR-561, hsa-miR-562, hsa-miR-563, hsa-miR-564, hsa-miR-566, hsa-miR-567, hsa-miR-568, hsa-miR-569, hsa-miR-570, hsa-miR-571, hsa-miR-572, hsa-miR-573, hsa-miR-574-3p, hsa-miR-574-5p, hsa-miR-575, hsa-miR-576-3p, hsa-miR-576-5p, hsa-miR-577, hsa-miR-578, hsa-miR-579, hsa-miR-580, hsa-miR-581, hsa-miR-582-3p, hsa-miR-582-5p, hsa-miR-583, hsa-miR-584, hsa-miR-585, hsa-miR-586, hsa-miR-587, hsa-miR-588, hsa-miR-589, hsa-miR-589*, hsa-miR-590-3p, hsa-miR-590-5p, hsa-miR-591, hsa-miR-592, hsa-miR-593, hsa-miR-593*, hsa-miR-595, hsa-miR-596, hsa-miR-597, hsa-miR-598, hsa-miR-599, hsa-miR-600, hsa-miR-601, hsa-miR-602, hsa-miR-603, hsa-miR-604, hsa-miR-605, hsa-miR-606, hsa-miR-607, hsa-miR-608, hsa-miR-609, hsa-miR-610, hsa-miR-611, hsa-miR-612, hsa-miR-613, hsa-miR-614, hsa-miR-615-3p, hsa-miR-615-5p, hsa-miR-616, hsa-miR-616*, hsa-miR-617, hsa-miR-618, hsa-miR-619, hsa-miR-620, hsa-miR-621, hsa-miR-622, hsa-miR-623, hsa-miR-624, hsa-miR-624*, hsa-miR-625, hsa-miR-625*, hsa-miR-626, hsa-miR-627, hsa-miR-628-3p, hsa-miR-628-5p, hsa-miR-629, hsa-miR-629*, hsa-miR-630, hsa-miR-631, hsa-miR-632, hsa-miR-633, hsa-miR-634, hsa-miR-635, hsa-miR-636, hsa-miR-637, hsa-miR-638, hsa-miR-639, hsa-miR-640, hsa-miR-641, hsa-miR-642, hsa-miR-643, hsa-miR-644, hsa-miR-645, hsa-miR-646, hsa-miR-647, hsa-miR-648, hsa-miR-649, hsa-miR-650, hsa-miR-651, hsa-miR-652, hsa-miR-653, hsa-miR-654-3p, hsa-miR-654-5p, hsa-miR-655, hsa-miR-656, hsa-miR-657, hsa-miR-658, hsa-miR-659, hsa-miR-660, hsa-miR-661, hsa-miR-662, hsa-miR-663, hsa-miR-663b, hsa-miR-664, hsa-miR-664*, hsa-miR-665, hsa-miR-668, hsa-miR-671-3p, hsa-miR-671-5p, hsa-miR-675, hsa-miR-7, hsa-miR-708, hsa-miR-708*, hsa-miR-7-1*, hsa-miR-7-2*, hsa-miR-720, hsa-miR-744, hsa-miR-744*, hsa-miR-758, hsa-miR-760, hsa-miR-765, hsa-miR-766, hsa-miR-767-3p, hsa-miR-767-5p, hsa-miR-768-3p, hsa-miR-768-5p, hsa-miR-769-3p, hsa-miR-769-5p, hsa-miR-770-5p, hsa-miR-802, hsa-miR-873, hsa-miR-874, hsa-miR-875-3p, hsa-miR-875-5p, hsa-miR-876-3p, hsa-miR-876-5p, hsa-miR-877, hsa-miR-877*, hsa-miR-885-3p, hsa-miR-885-5p, hsa-miR-886-3p, hsa-miR-886-5p, hsa-miR-887, hsa-miR-888, hsa-miR-888*, hsa-miR-889, hsa-miR-890, hsa-miR-891a, hsa-miR-891b, hsa-miR-892a, hsa-miR-892b, hsa-miR-9, hsa-miR-9*, hsa-miR-920, hsa-miR-921, hsa-miR-922, hsa-miR-923, hsa-miR-924, hsa-miR-92a, hsa-miR-92a-1*, hsa-miR-92a-2*, hsa-miR-92b, hsa-miR-92b*, hsa-miR-93, hsa-miR-93*, hsa-miR-933, hsa-miR-934, hsa-miR-935, hsa-miR-936, hsa-miR-937, hsa-miR-938, hsa-miR-939, hsa-miR-940, hsa-miR-941, hsa-miR-942, hsa-miR-943, hsa-miR-944, hsa-miR-95, hsa-miR-96, hsa-miR-96*, hsa-miR-98, hsa-miR-99a, hsa-miR-99a*, hsa-miR-99b, and hsa-miR-99b*.

A miRNA inhibits the function of the mRNAs it targets and, as a result, inhibits expression of the polypeptides encoded by the mRNAs. Thus, blocking (partially or totally) the activity of the miRNA (e.g., silencing the miRNA) can effectively induce, or restore, expression of a polypeptide whose expression is inhibited (derepress the polypeptide). In one embodiment, derepression of polypeptides encoded by mRNA targets of a miRNA is accomplished by inhibiting the miRNA activity in cells through any one of a variety of methods. For example, blocking the activity of a miRNA can be accomplished by hybridization with a small interfering nucleic acid (e.g., antisense oligonucleotide, miRNA sponge, TuD RNA) that is complementary, or substantially complementary to, the miRNA, thereby blocking interaction of the miRNA with its target mRNA. As used herein, an small interfering nucleic acid that is substantially complementary to a miRNA is one that is capable of hybridizing with a miRNA, and blocking the miRNA's activity. In some embodiments, a small interfering nucleic acid that is substantially complementary to a miRNA is a small interfering nucleic acid that is complementary to the miRNA at all but 1, 2, 3, 4, 5, 6, 7, 8, 9, 10, 11, 12, 13, 14, 15, 16, 17, or 18 bases. In some embodiments, a small interfering nucleic acid sequence that is substantially complementary to a miRNA, or is a small interfering nucleic acid sequence that is complementary to the miRNA with at least one base.

A “miRNA Inhibitor” is an agent that blocks miRNA function, expression and/or processing. For instance, these molecules include but are not limited to microRNA specific antisense, microRNA sponges, tough decoy RNAs (TuD RNAs) and microRNA oligonucleotides (double-stranded, hairpin, short oligonucleotides) that inhibit miRNA interaction with a Drosha complex. MicroRNA inhibitors can be expressed in cells from a transgenes of a rAAV vector, as discussed above. MicroRNA sponges specifically inhibit miRNAs through a complementary heptameric seed sequence (Ebert, M. S. Nature Methods, Epub Aug. 12, 2007). In some embodiments, an entire family of miRNAs can be silenced using a single sponge sequence. TuD RNAs achieve efficient and long-term-suppression of specific miRNAs in mammalian cells (See, e.g., Takeshi Haraguchi, et al., Nucleic Acids Research, 2009, Vol. 37, No. 6 e43, the contents of which relating to TuD RNAs are incorporated herein by reference). Other methods for silencing miRNA function (derepression of miRNA targets) in cells will be apparent to one of ordinary skill in the art.

In some embodiments, the cloning capacity of the recombinant RNA vector may limit a desired coding sequence and may require the complete replacement of the virus's 4.8 kilobase genome. Large genes may, therefore, not be suitable for use in a standard recombinant AAV vector, in some cases. The skilled artisan will appreciate that options are available in the art for overcoming a limited coding capacity. For example, the AAV ITRs of two genomes can anneal to form head to tail concatamers, almost doubling the capacity of the vector. Insertion of splice sites allows for the removal of the ITRs from the transcript. Other options for overcoming a limited cloning capacity will be apparent to the skilled artisan.

### Administration

The rAAVs may be delivered to a subject in compositions according to any appropriate methods known in the art. The rAAV, preferably suspended in a physiologically compatible carrier (e.g., in a composition), may be administered to a subject, e.g., host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, administration is performed using intra-articular injection. In some embodiments, administration is performed using direct injection to a joint. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to a joint of a subject. “Joint” is intended to include all cells and tissue of joint space (e.g., knee joint, shoulder joint, elbow joint, hip joint, ankle joint, spinal joint, finger joint, or toe joint). Thus, the term includes, but is not limited to, skin, muscle, bone cells, articular cartilage, meniscus, synovium, ligament tissues and the like. Recombinant AAVs may be delivered directly to a joint by injection into an artery, a vein, the bloodstream, the space within a joint, etc.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intra-articular injection, intraocular, intravenous, intramuscular, subcutaneous, intradermal, intratumoral, intracranial (e.g., intrahippocampal), and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An effective amount of an rAAV is an amount sufficient to target infect an animal, target a desired tissue. In some embodiments, an effective amount of an rAAV is an amount sufficient to produce a stable somatic transgenic animal model. The effective amount will depend primarily on factors such as the species, age, weight, health of the subject, and the tissue to be targeted, and may thus vary between animals or tissues. For example, an effective amount of the rAAV is generally in the range of from about 1 ml to about 100 ml of solution containing from about 10⁹ to 10¹⁶ genome copies. In some embodiments the rAAV is administered at a dose of 10¹⁰, 10¹¹, 10¹², 10¹³, 10¹⁴, or 10¹⁵ genome copies per subject. In some embodiments the rAAV is administered at a dose of 10¹⁰, 10¹¹, 10¹², 10¹³, or 10¹⁴ genome copies per kg. In some cases, a dosage between about 10¹¹ to 10¹² rAAV genome copies is appropriate. In certain embodiments, 10¹² rAAV genome copies is effective to target heart, liver, and pancreas tissues. In some cases, stable transgenic animals are produced by multiple doses of an rAAV.

In some embodiments, rAAV compositions are formulated to reduce aggregation of AAV particles in the composition, particularly where high rAAV concentrations are present (e.g., ˜10¹³ GC/ml or more). Methods for reducing aggregation of rAAVs are well-known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intraopancreatically, intranasally, parenterally, intravenously, intracranially (e.g., intrahippocampally), intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered trangenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500.ANG., containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### EXAMPLES

### Example 1

Recombinant adeno-associated viruses (rAAVs) including AAV2 have emerged as a possible vector for delivery of transgenes to arthritic tissues and joints because of their ability to transduce non-dividing cells, long-term transgene expression without the need for viral genome integration, lack of known disease association, and low immunogenicity. Over the last decade, researchers have made advances in rAAV vector design to improve tissue specificity, transduction efficacy, and eliminate immunogenicity. An AAV2 vector is currently used in Phase I clinical trials in the United States for OA gene therapy. However, pre-existing neutralizing antibodies (NAbs) to AAV2 may limit patient access, since patients who have NAbs against the AAV gene therapy vector are eliminated from the gene therapy. It is therefore important to expand the diversity of AAV capsid variants for rAAV gene therapy vectors. Moreover, novel vectors and capsids may confer higher transduction levels in joints (e.g., the knee joint) with longer periods and lower immune responses will be powerful gene therapy tools for OA.

Novel AAV capsids that can transduce at higher efficiencies than AAV2 in an arthritic knee joint were identified. A high-throughput sequencing method identified forty-two AAV2 capsid variants (a capsid protein having the sequence set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84).

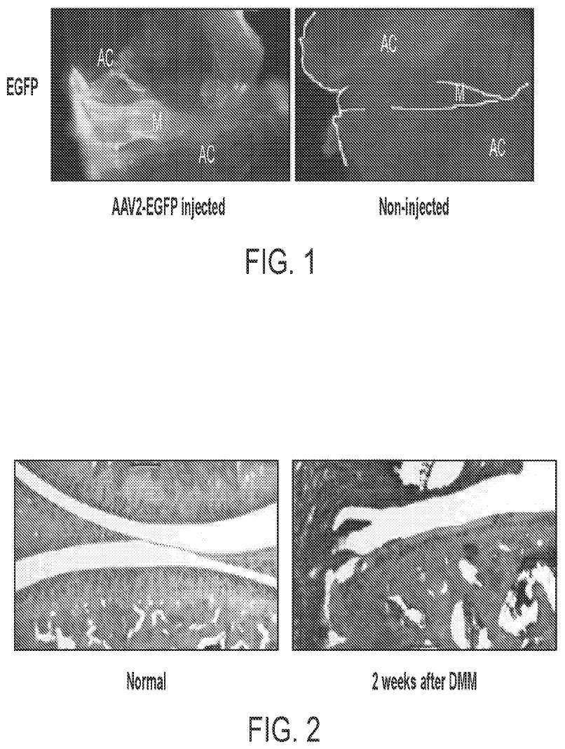

It was confirmed that rAAV2 can transduce the knee joint of a mouse by intra-articular injection of an rAAV2 comprising a transgene encoding enhanced green florescent protein (EGFP) (FIG. 1). One week after injection, the mouse knee joint was harvested and expression of the fluorescent EGFP protein was observed in the joint space (articular cartilage and meniscus) with a fluorescent microscope.

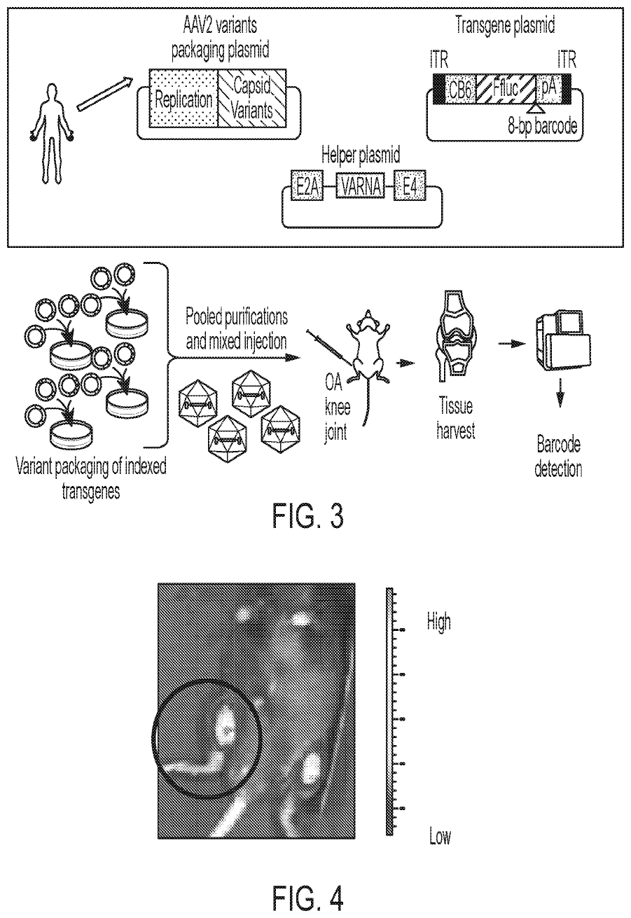

A mouse model of osteoarthritis was generated by surgical destabilization of the medial meniscus (DMM). Two weeks after the surgical destabilization, disorganization and cell death of articular cartilage were observed (FIG. 2). DMM mimics clinical meniscal injury, a known provoking factor for the development of OA, and allows the study of structural and biological changes throughout the progression of OA. The transduction efficacy of AAV2 variants were tested in the mouse model of OA. Firefly luciferase (Fluc) transgenes that harbor unique 8-bp barcode in the 3′-UTR were packaged into each of the AAV2 variants and a mixed purification to generate an AAV2-variant library was generated to test as a single injection into the mouse OA knee joint and unoperated knee joints (FIG. 3). The AAV2-variant library was prepared using triple plasmid transfection of HEK293 cells and subsequently injected into OA mice by intra-articular injection two weeks after DMM surgery. The AAV2-variant library was similarly injected into healthy mice (i.e., mice that have not had DMM surgery).

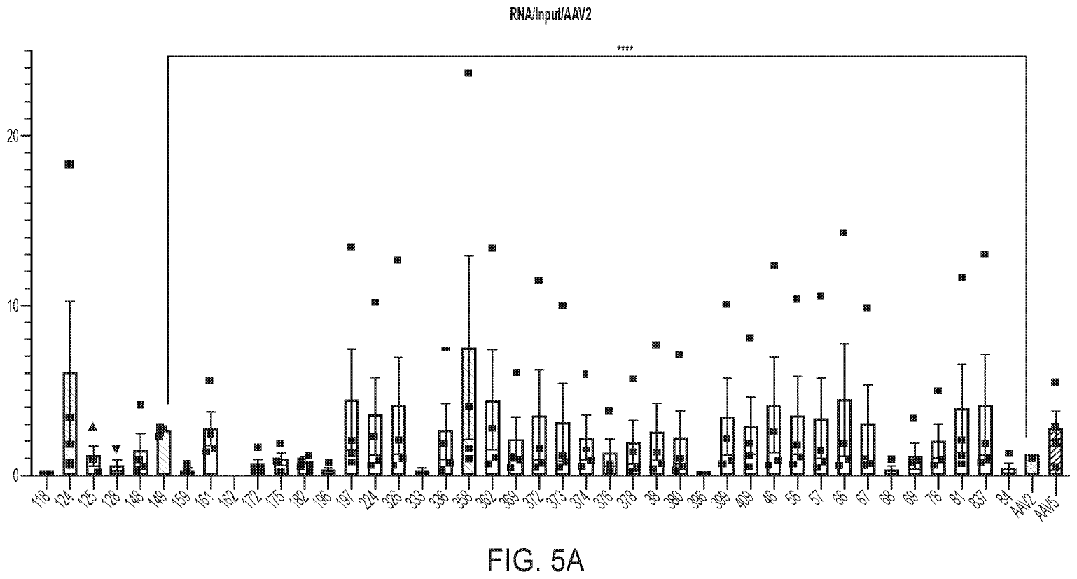

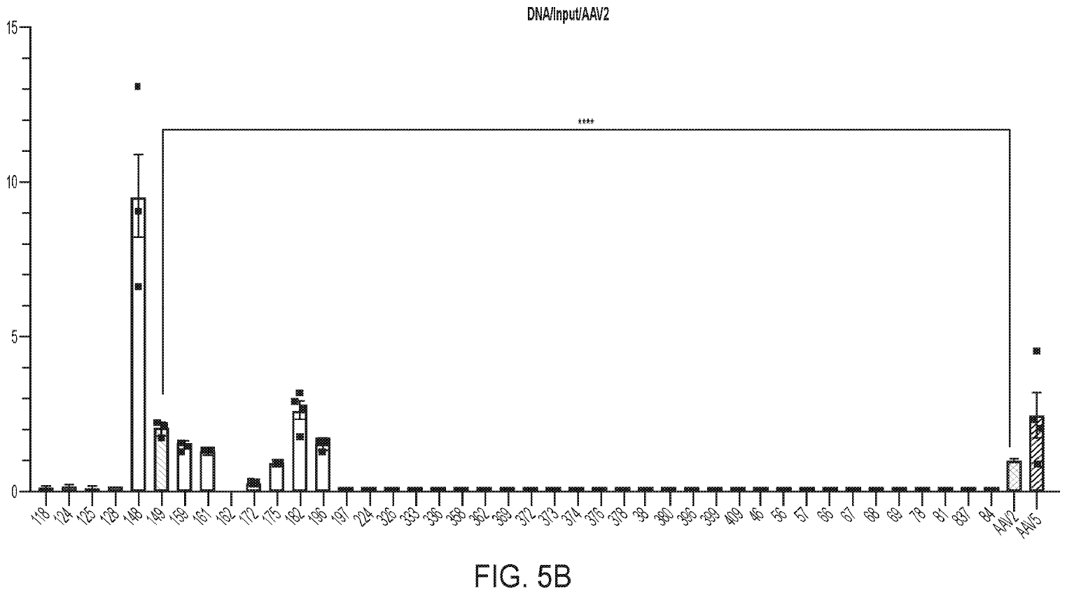

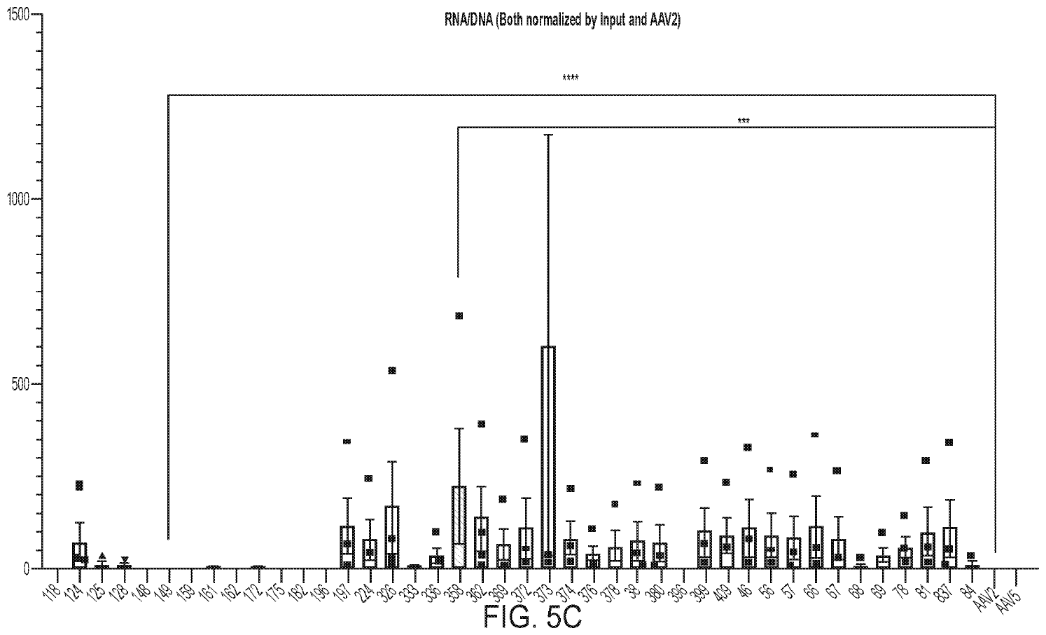

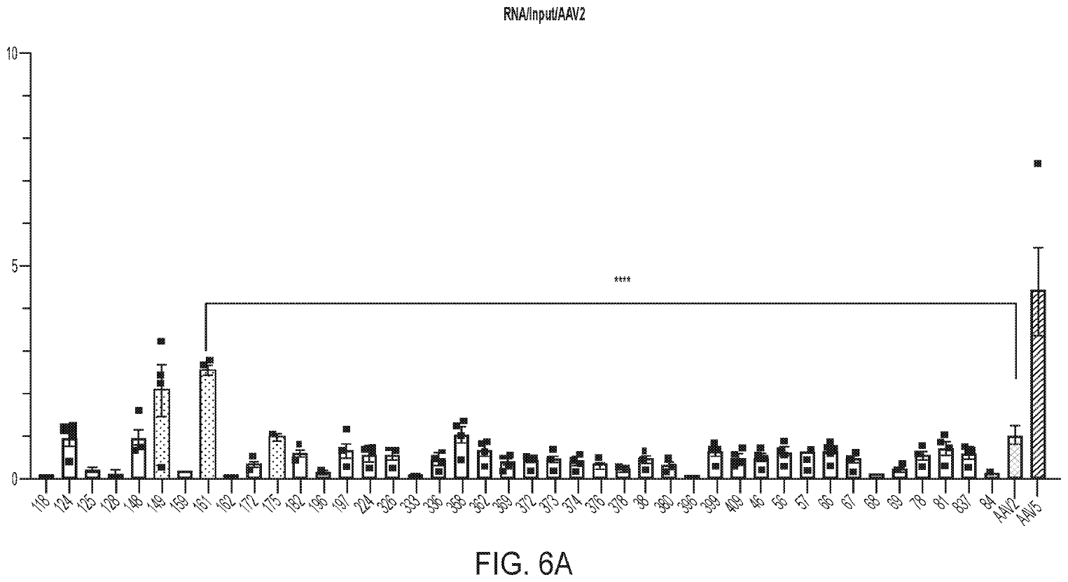

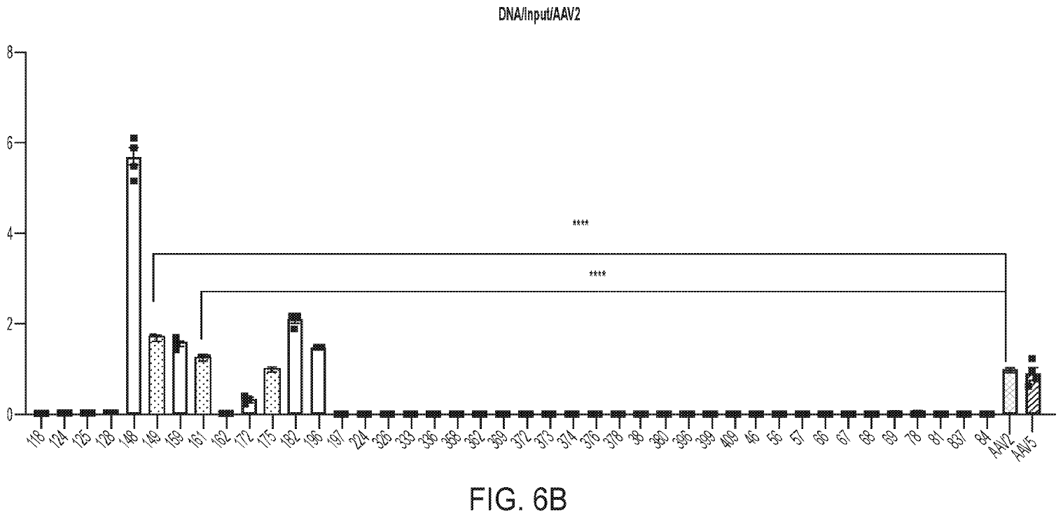

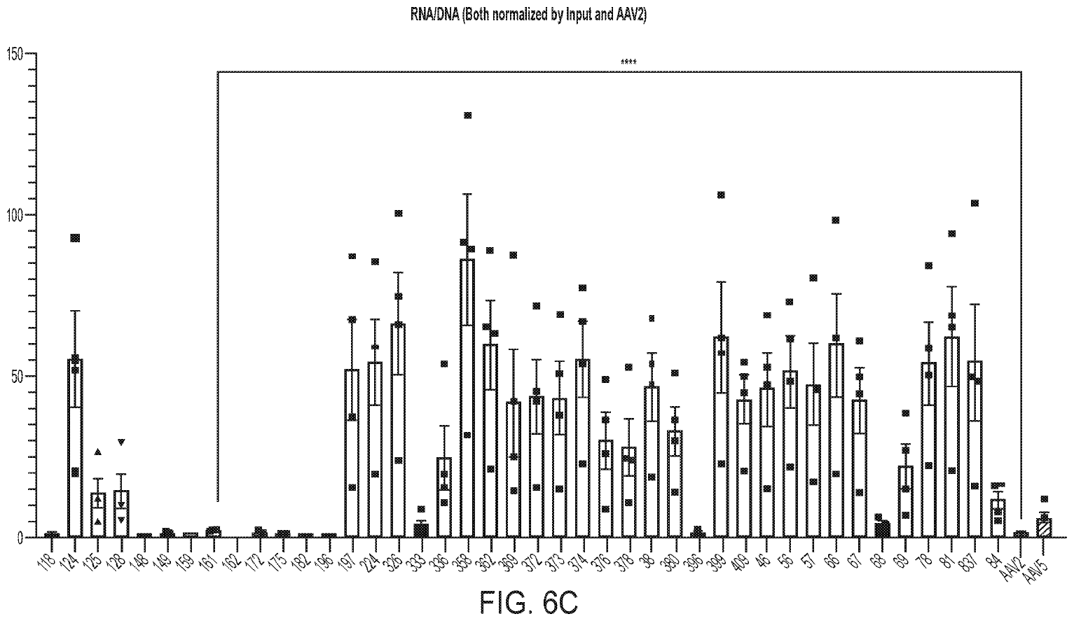

Mice were sacrificed two weeks after the injection and knee joints were harvested. Whole DNA and RNA from knee joints were extracted to detect and quantify the distribution of barcoded vector genomes and firefly luciferase transcripts. The transduction of the variants in OA and intact knee joints was observed using bioluminescence analysis (FIG. 4) to quantify the total amount of expressed firefly luciferase. Both type of the knee joints were dissected, and the skin, muscle, and bone tissues were carefully removed. RNA and DNA were isolated from the knee joints that contain only the articular cartilage, meniscus, synovium, and ligament tissues and/or bone around the knee space. Isolated RNA and DNA was subjected to library construction and deep sequencing analysis (N=4). The transduction efficiencies of each individual AAV2 variant was determined by quantifying the relative abundances of individual barcode sequences detected in the method. Based on this analysis, three capsids that presented transduction levels that were equivalent to or higher than those conferred by AAV2 in both OA (FIG. 5) and intact normal (FIG. 6) knees for further characterization.

FIG. 5 shows the sequencing reads of the 8 bp barcodes of the AAV2 variants which transduced mouse OA knee joints. The read count of each variant's RNA transcript is shown relative to input normalized to AAV2 levels (Input: genomic titer of each variant in the injected mixture). Data are shown as the mean±standard error (***P<0.0005, ***P<0.0001) FIG. 6 shows the sequencing reads of the 8 bp barcodes of the AAV2 variants which transduced mouse normal knee joints. The read count of each variant's RNA transcript is shown relative to input normalized to AAV2 levels (Input: genomic titer of each variant in the injected mixture). Data are shown as the mean±standard error (***P<0.0005, ****P<0.0001).

One variant (AAVv149; a capsid protein having the sequence set forth in SEQ ID NO: 149) transduced both healthy and OA knee joints approximately 2.0-fold better than AAV2, while four variants (AAVv124, AAVv358, AAVv161, AAVv175; a capsid protein having the sequence set forth in SEQ ID NOs: 124, 358, 161, and 175) exhibited mutual exclusive tropism profiles. These AAV2 variants are anticipated to be effective gene therapy vectors for delivering transgenes to a joint of a subject and reducing inflammation in the joint of the subject.

TABLE 1
Selected capsid variants of the disclosure

| Capsid | SEQ ID | |
|---|---|---|
| variant | NO: | Amino acid sequence |
| v149 | 149 | MAADGYLPDWLEDTLSEGIRQWWKLKPGPPPPKPAGRHKDDSRGLVLPGYKYLG |
| | | PFNGLDKGEPVNEADAAALEHDKAYDRQLDSGDNPYLKYNHADAEFQERLKEDT |
| | | SFGGNLGRAVFQAKKRALEPLGLVEEPVKTAPGKKRPVEHSPVEPDSSSGTGKAGQ |
| | | QPARKRLNFGQTGDADSVPDPQPLGQPPAAPSGLGTNTMATGSGAPMADNNEGA |
| | | DGVGNSSGNWHCDSTWMGDRVITTSTRTWALPTYNNHLYKQISSQSGASNDNHY |
| | | FGYSTPWGYFDFNRFHCHFSPRDWQRLINNNWGFRPKRLNFKLFNIQVKEVTQND |
| | | GTTTIANNLTSTVQVFTDSEYQLPYVLGSAHQGCLPPFPADVFMVPQYGYLTLNNG |
| | | SQAVGRSSFYCLEYFPSQMLRTGNNFTFSYTFEDVPFHSSYAHSQSLDRLMNPLIDQ |
| | | YLYYLSRTNTPSGTTTQSRLQFSQAGASDIRDQSRNWLPGPCYRQQRVSKTSADNN |
| | | NSEYSWTGATKYHLNGRDSLVNPGPAMASHKDDEEKFFPQSGVLIFGKQGSEKTN |
| | | VDIEKVMITDEEEIRTTNPVATEQYGSVSTNLQRGNRQAATADVNTQGVLPGMVW |
| | | QDRDVYLQGPIWAKIPHTDGHFHPSPLMGGFGLKHPPPQILIKNTPVPANPSTTFSA |
| | | AKFASFITQYSTGQVSVEIEWELQKENSKRWNPEIQYTSNYNKSVNVDFTVDTNGV |
| | | YSEPRPIGTRYLTRNL |
| v124 | 124 | MAADGYLPDWLEDTLSEGIRQWWKLKPGPPPPKPAERHKDDSRGLVLPGYKYLG |
| | | PFNGLDKGEPVNEADAAALEHDKAYDRQLDSGDNPYLKYNHADAEFQERLKEDT |
| | | SFGGNLARAVFQAKKRVLEPLGLVEEPVKTAPGKKRPVEHSPAEPDSSSGTGKSGQ |
| | | QPARKRLNFGQTGDADSVPDPQPLGQPPAAPSGLGTNTMASGSGAPMADNNEGA |
| | | DGVGNSSGNWHCDSTWMGDRVITTSTRTWALPTYNNHLYKQISSQSGASNDNHY |
| | | FGYSTPWGYFDFNRFHCHFSPRDWQRLINNNWGFRPKRLNFKLFNIQVKEVTQND |
| | | GTTTIANNLTSTVQVFTDSEYQLPYVLGSAHQGCLPPFPADVFMVPQYGYLTLNNG |
| | | SQAVGRSSFYCLEYFPSQMLRTGNNFTFSYTFEDVPFHSSYAHSQSLDRLMNPLIDQ |
| | | YLYYLSRTNTPSGTTTQSRLRFSQAGASDIRDQSRNWLPGPCYRQQRVSKTAADNN |
| | | NSDYSWTGATKYHLNGRDSLVNPGTAMASHKDDEEKYFPQSGVLIFGKQDSGKT |
| | | NVDIERVMITDEEEIRTTNPVATEQYGSVSTNLQSGNTQAATSDVNTQGVLPGMV |
| | | WQDRDVYLQGPIWAKIPHTDGHFHPSPLMGGFGLKHPPPQILIKNTPVPANPSTTFS |
| | | AAKFASFITQYSTGQVSVEIEWELQKENSKRWNPEIQYTSNYNKSVNVDFTVDING |
| | | VYSEPRPIGTRYLTRNL |
| v358 | 358 | MAADGYLPDWLEDTLSEGIRQWWKLKPGPPPPKPAERHKDDSRGLVLPGYKYLG |
| | | PFNGLDKGEPVNEADAAALEHDKAYDRQLDSGDNPYLKYNHADAEFQERLKEDT |
| | | SFGGNLGRAVFQAKKRVLEPLGLVEEPVKTAPGKKRPVEHSPVESDSSSGTGKAGQ |
| | | QPARKRLNFGQTGDADSVPDPQPLGQPPAAPSGLGTNTMATGSGAPMADNNEGA |
| | | DGVGNSSGNWHCDSTWMGDRVITTSTRTWALPTYNNHLYKQISSQSGASNDNHY |
| | | FGYSTPWGYFDFNRFHCHFSPRDWQRLINNNWGFRPKRLSFKLFNIQVKEVTQND |
| | | GTTTIANNLTSTVQVFTDSEYQLPYVLGSAHQGCLPPFPADVFMVPQYGYLTLNNG |
| | | SQAVGRSSFYCLEYFPSQMLRTGNNFTFSYTFEDVPFHSSYAHSQSLDRLMNPLIDQ |
| | | YLYYLSKTNAPSGTTTMSRLQFSQAGASDIRDQSRNWLPGPCYRQQRVSKTAADN |
| | | NNSDYSWTGATKYHLNGRDSLVNPGPAMASHKDDEEKYFPQSGVLIFGKQGSNK |
| | | TNVDIEKVMITDEEEIRTTNPVATEQYGSVSTNLQSGNTQAATSDVNTQGVLPGMV |
| | | WQDRDVYLQGPIWAKIPHTDGHFHPSPLMGGFGLKHPPPQILIKNTPVPANPSTTFS |
| | | AAKFASFITQYSTGQVSVEIEWELQKENSKRWNPEIQYTSNYNKSVNVDFTVDING |
| | | VYSEPRPIGTRYLTRNL |
| v161 | 161 | MAADGYLPDWLEDTLSEGIRQWWKLKPGPPPPKPAERHKDDSRGLVLPGYKYLG |
| | | PFNGLDKGEPVNEADAAALEHDKAYDRQLDSGDNPYLKYNHADAEFQERLKEDT |
| | | SFGGNLGRAVFQAKKRALEPLGLVEEPVKTAPGKKRPVEHSPVEPDSSSGTGKAGQ |
| | | QPARKRLNFGQTGDADSVPDPQPLGQPPAAPSGLGTNTMATGSGAPMADNNEGA |
| | | DGVGNSSGNWHCDSTWMGDRVITTSTRTWALPTYNNHLYKQISSQSGASNDNHY |
| | | FGYSTPWGYFDFNRFHCHFSPRDWQRLINNNWGFRPKRLNFKLFNIQVKEVTQND |
| | | GTTTIANNLTSTVQVFTDSEYQLPYVLGSAHQGCLPPFPADVFVVPQYGYLTLNNG |
| | | SQAVGRSSFYCLEYFPSQMLRTGNNFTFSYTFEDVPFHSSYAHSQSLDRLMNPLIDQ |
| | | YLYYLSRTNTPSGTTTQSRLQFSQAGASDIRDQSRNWLPGPCYRQQRVSKTSADNN |
| | | NSEYSWTGATKYHLNGRDSLVNPGPAMASHKDDEEKFFPQSGVLIFGKQGSEKTN |
| | | VDIEKVMITDEEEIRTTNPVATEQYGSVSTNLQRGNRQAATADVNTQGVLPGMVW |
| | | QDRDVYLQGPIWAKIPHTDGHFHPSPLMGGFGLKHPPPQILIKNTPVPANPSTTFSA |
| | | AKFASFITQYSTGQVSVEIEWELQKENSKRWNPEIQYTSNYNKSVNVDFTVDTNGV |
| | | YSEPRPIGTRYLTRNL |
| v175 | 175 | MAADGYLPDWLEDTLSEGIRQWWKLKPGPPPPKPAERHKDDSRGLVLPGYKYLG |
| | | PFNGLDKGEPVNEADAAALEHDKAYDRQLDSGDNPYLKYNHADAEFQERLKEDT |
| | | SFGGNLGRAVFQAKKRALEPLGLVEEPVKTAPGKKRPVEHSPVEPDSSSGTGKAGQ |
| | | QPARKRLNFGQTGDADSVPDPQPLGQPPAAPSGLGTNTMATGSGAPMADNNEGA |
| | | DGVGNSSGNWHCDSTWMGDRVITTSTRTWALPTYNNHLYKQISSQSGASNDNHY |
| | | FGYSTPWGYFDFNRFHCHFSPRDWQRLINNNWGFRPKRLNFKLFNIQVKEVTQND |
| | | GTTTIANNLTSTVQVFTDSEYQLPYVLGSAHQGCLPPFPADVFMVPQYGYLTLNNG |
| | | SQAVGRSSFYCLEYFPSQMLRTGNNFTFSYTFEDVPFHSSYAHSQSLDRLMNPLIDQ |
| | | YLYYLSRTNTPSGTTTQSRLQFSQAGASDIRDQSRNWLPGPCYRQQRVSKTSADNN |
| | | NSEYSWTGATKYHLNGRDSLVNPGPAMASHKDDEEKFFPQSGVLIFGKQGSEKTN |
| | | VDIEKVMITDEEEIRTTNPVATEQYGSVSTNLQRGNRQAATADVNTQGVLPGTVW |
| | | QDRDVYLQGPIWAKIPHTDGHFHPSPLMGGFGLKHPPPQILIKNTPVPANPSTTFSA |
| | | AKFASFITQYSTGQVSVEIEWELQKENSKRWNPEIQYTSNYNKSVNVDFTVDTNGV |
| | | YSEPRPIGTRYLTRNL |

### Example 2

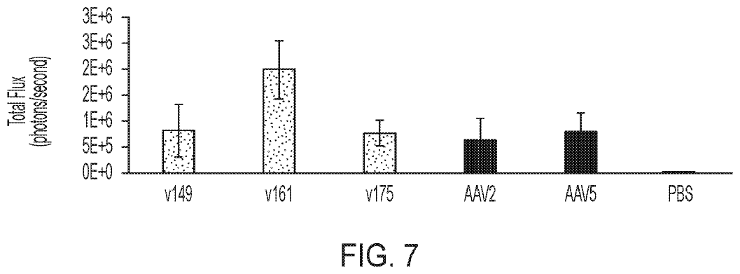

AAV2 capsid variants of the disclosure are able to transduce normal mouse knees. Three variants (v149 capsid variant comprising the amino acid sequence set forth in SEQ ID NO: 149; v161 capsid variant comprising the amino acid sequence set forth in SEQ ID NO: 161; and v175 capsid variant comprising the amino acid sequence set forth in SEQ ID NO: 175), AAV2, and AAV5 were packaged with firefly luciferase expression vectors (scAAV.CB6-FLuc) and injected into mouse knees at 1×10¹¹ gene copies per mouse. Mice were imaged live after two weeks post-injection to show transgene expression differences. Luminescence of the firefly luciferase (representative of total flux) was quantified from the images (FIG. 7). The v149 and v175 capsid variants provided a similar level of transgene expression in the mouse knees relative to the AA2 and AAV5 capsid proteins. The v161 capsid variant conferred a greater than two-fold increase in transduction relative to the AAV2 and AAV5 capsid proteins. These data demonstrate that the AAV2 capsid variants of the disclosure are capable of transducing knee joints in vivo.

### Example 3

The ability of the v149 capsid variant (comprising the amino acid sequence set forth in SEQ ID NO: 149) to transduce knee joints was assessed. The v149 capsid protein was packaged with enhanced green flurescent protein (Egfp) expression vectors (scAAV-CB6-PI-Egfp) and injected into mouse knees at 1×10¹⁰ gene copies per knee. After one month post-injection, mice were sacrificed and knee joints were collected for cryosectioning and imaging. Mice injected with phosphate-buffered saline and the AAV2 capsid protein packaged with enhanced green flurescent protein (Egfp) expression vectors (scAAV-CB6-PI-Egfp) were used as controls. The data from the cryosectioning and imaging demonstrate that the v149 capsid variant is able to transduce knee joints to deliver the Egfp transgene (for expression of Egfp in the knee joints) at comparable or improved levels relative to the AAV2 capsid protein.

1. A method for delivering a transgene to a joint in a subject, the method comprising administering to the subject a recombinant adeno-associated virus (rAAV) comprising:
(i) an isolated nucleic acid comprising a transgene encoding one or more gene products of interest; and
(ii) an adeno-associated acid (AAV) capsid protein having the sequence set forth in any one of SEQ ID NOs: 118, 124, 125, 128, 148, 149, 159, 161, 162, 172, 175, 182, 196, 197, 224, 326, 333, 336, 358, 362, 369, 372, 373, 374, 376, 378, 38, 380, 396, 399, 409, 46, 56, 57, 66, 67, 68, 69, 78, 81, 837, or 84.

2. The method of claim 1, wherein the administration comprises intra-articular injection.

3. The method of claim 1, wherein the joint is a knee joint, shoulder joint, elbow joint, hip joint, ankle joint, spinal joint, finger joint, or toe joint.

4. The method of claim 1, wherein the joint comprises skin, muscle, bone cells, articular cartilage, meniscus, synovium, and/or ligament tissues.

5. The method of claim 1, wherein the subject is a human.

6. The method of claim 1, wherein the subject is characterized by production of anti-AAV2 antibodies prior to the administration.

7. The method of claim 6, wherein after administration of the rAAV, the subject does not elicit a neutralizing immune response against the rAAV.

8. The method of claim 1, wherein the isolated nucleic acid comprises AAV inverted terminal repeats (ITRs) flanking the transgene.

9. The method of claim 1, wherein the nucleic acid sequence encoding the one or more gene products is operably linked to a promoter.

10. The method of claim 1, wherein the one or more gene products comprise a protein or an inhibitory nucleic acid.

11. The method of claim 1, wherein the AAV capsid protein has the sequence set forth in any one of SEQ ID NOs: 124, 149, 161, 175, or 358.

12. The method of claim 1, wherein the transgene is expressed in the joint at elevated levels relative to a wild-type AAV2 capsid protein.

13. The method of claim 1, wherein the one of more gene products is an anti-inflammatory gene product, optionally wherein the anti-inflammatory gene product is selected from the group consisting of: Insulin-like growth factor 1 (IGF-1), IL-1ra, IL-4, IL-6, IL-10, IL-11, IL-13, TGF-β, TNF receptor p55 (sTNFRI or sTNFRp55), TNF receptor p75 (sTNFRII or sTNFRP75), IL-1 receptor type 2 (sIL-1RII), membrane-bound IL-1 receptor type 2 (mIL-1RII), and IL-18 binding protein (IL-18BP).

14. The method of claim 1, the one of more gene products inhibit the expression or function of a pro-inflammatory gene, optionally wherein the pro-inflammatory gene is IL-1, tumor necrosis factor (TNF), IL-12, IL-18, interferon gamma (IFNγ), and granulocyte-macrophage colony stimulating factor (GM-CSF).

15-21. (canceled)

22. The method of claim 8, wherein the AAV inverted terminal repeats (ITRs) flanking the transgene are AAV2 ITRs.

23. (canceled)

24. The method of claim 1 wherein the one or more gene products comprise a protein or an inhibitory nucleic acid.

25-29. (canceled)

30. The method of claim 1, wherein the subject has osteoarthritis (OA) or rheumatoid arthritis (RA) in the joint.
